(19) United States
(12) Patent Application Publication
(10) Pub. No.: US 2022/0218710 A1
(43) Pub. Date: Jul. 14, 2022

(54) Inhibition of Neddylation for Treatment of MS

(71) Applicant: The Regents of the University of California, Oakland, CA (US)

(72) Inventors: SERGIO BARANZINI, San Francisco, CA (US); ANNE-KATRIN PROEBSTEL, Basel (CH); KICHEOL KIM, San Francisco, CA (US)

(73) Assignee: The Regents of the University of California, Oakland, CA (US)

(21) Appl. No.: 17/608,134

(22) PCT Filed: Apr. 30, 2020
(86) PCT No.: PCT/US2020/030776

§ 371 Date: Nov. 1, 2021

Related U.S. Application Data
(60) Provisional application No. 62/841,824, filed on May 1, 2019.

(51) Int. Cl. A61K 31/519; A61P 25/00

(52) U.S. Cl. CPC A61K 31/519; A61P 25/00

(57) ABSTRACT

The use of neddylation inhibitors to treat immune related conditions is described. Dysregulated immune and inflammatory responses underlie a number of conditions, including multiple sclerosis. Neddylation is involved in processes underlying the activation, activity, and proliferation of immune cells that drive these conditions, including CD4+ T cells. Inhibition of neddylation thus provides a target for intervention to prevent and treat such conditions. Inhibition of E1 NEDD8 activating enzymes, such as NAE may be utilized in treatment, for example by pevonedistat or other NAE inhibitors.

### CROSS-REFERENCE TO RELATED APPLICATIONS: This application is a 35

USC § 371 national stage application of PCT/US2020/030776, entitled “Inhibition of Neddylation for Treatment of MS,” filed Apr. 30, 2020, which claims the benefit of priority to U.S. Provisional Patent Application Ser. No. 62/841,824 entitled “Inhibition of Neddylation for Treatment of MS,” filed May 1, 2019, the contents of which applications are hereby incorporated by reference.

### STATEMENT REGARDING FEDERALLY SPONSORED RESEARCH OR DEVELOPMENT

This invention was made with government support under grant number R01 NS088155 awarded by the National Institutes of Health. The government has certain rights in the invention.

### BACKGROUND OF THE INVENTION

Autoimmune conditions of the central nervous system (CNS) are prevalent ailments with widespread morbidity and mortality. For example, multiple sclerosis (MS) is a chronic autoimmune condition of the central nervous system characterized by demyelination and neurodegeneration leading to permanent clinical disability. While it is clear that both genetic and environmental factors contribute to MS pathogenesis, its etiology remains elusive. With the exception of anti-B cell therapies, current immunomodulatory therapies are only partially effective.

Over the past decade, genome-wide association studies identified many independent risk loci in MS, most of which harbor genes primarily expressed in immune cells such as T cells and monocytes. Previous gene expression studies used whole blood or peripheral blood mononuclear cells (PBMC), mostly searching for dysregulated MS pathogenic pathways and biomarkers of disease progression. However, this approach only detects the strongest signals due to highly variable gene expression across the complex mixture of cell types present in each sample. Accordingly, there is a need in the art for finer resolution of gene expression patterns within different sets of immune cells in order to identify pathways that are dysregulated in MS and other inflammatory conditions of the central nervous system, in order to identify novel therapeutic targets and interventions.

### SUMMARY OF THE INVENTION

In order to increase the sensitivity of gene expression analysis in MS and other conditions, the inventors of the present disclosure performed cell-type-specific RNA-seq from FACS-sorted specific immune cell populations, thus providing greatly enhanced signal-to-noise ratio. Total RNA was sequenced in FACS-sorted CD4+ T cells, CD8+ T cells, and CD14+ monocytes from MS subjects. As described in more detail in the Examples section below, by analysis of differentially expressed genes in subsets of immune cells in MS subject, the inventors of the present disclosure have determined that neddylation is a critical target in dysregulated immune cells. Furthermore, the inventors of the present disclosure demonstrated that inhibition of neddylation, for example, by inhibition of NAE (NEDD8-activating enzyme), provides therapeutic benefits in the treatment of MS and other conditions encompassing CNS inflammation or immune activity.

In a first aspect, the scope of the invention encompasses novel methods of treating CNS inflammation by disrupting post-translational protein modifications that underlie CNS inflammatory conditions. In one aspect, the therapeutic methods encompass the disruption of neddylation. In one embodiment, the disruption of neddylation is achieved by inhibition of NAE. In one embodiment, the inhibition of NAE1 is achieved by administration of pevonedistat or like compositions. In several implementations, the methods of the invention are directed to the treatment of various inflammatory conditions of the CNS. In a primary embodiment, the method is directed to the treatment of MS. The various embodiments of the invention are described in more detail in the following sections.

### BRIEF DESCRIPTION OF THE DRAWINGS

FIG. 1. FIG. 1 depicts a schematic overview of the RNA-seq experiments and transcriptome data analysis performed in the Examples.

FIG. 2. FIG. 2 depicts the neddylation pathway and effect in CD4+ T cells.

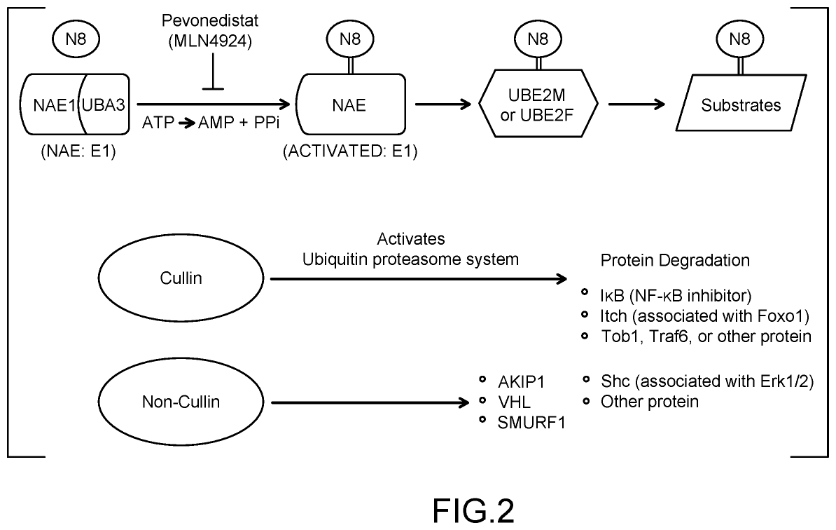

FIGS. 3A, 3B, and 3C. FIGS. 3A, 3B, and 3C depicts pevonedistat treatment effects in an MS animal model. FIG. 3A depicts the trial design: C57BL/6 mice were treated daily starting on Day−1 with either Pevonedistat (20 mg/kg) or placebo (n=10/group). The result shown here is one representative of two independent experiments. EAE was induced through active immunization with myelin oligodendrocyte (MOG) peptide 35-55 following standard protocols (triangles). Weights were taken on D−1, D7, and D14 (dots). Animals were scored daily. Mice were sacrificed and brain and spinal cords of were harbored on Day 16 at peak disease (n=4/group) (triangle). FIG. 3B depicts EAE severity: Diseases was assessed blinded using a 10-point scale from 0 to 5 (0=no symptoms, 5=death). Data shown here are from one representative of two experiments. FIG. 3C depicts mice weights over the course of the experiment. ****p-value<0.001, 2-way ANOVA test.

### DETAILED DESCRIPTION OF THE INVENTION

The scope of the invention encompasses numerous methods directed to the use of neddylation inhibitors for the maintenance of health, prevention of disease, and in therapeutic applications. In one aspect, the scope of the invention encompasses methods of treating immune-mediated conditions of the by the administration of neddylation inhibitors. The general method of the invention encompasses:

a method of treating an immune condition in a subject in need of treatment therefore by the administration of a pharmaceutically effective amount of a neddylation inhibitor.
In a related embodiment, the scope of the invention encompasses a neddylation inhibitor for use in a method of treating an immune condition, as defined below. In another related embodiment, the scope of the invention encompasses the use of a neddylation inhibitor in a method of making a medicament for the treatment of an immune condition. The various elements of the general method are described next.

Subjects. The methods disclosed herein will be directed to administration of neddylation inhibitors to subjects. The subject may be a human subject, for example, in some contexts, a patient. The subject may also comprise a non-human animal of any species, including test animals, veterinary subjects, pets, and livestock, for example, any of mice, rats, dogs, cats, sheep, goats, cows, pigs, horses, camels, non-human primates, or other animals. In certain embodiments, the subject of the method will be a subject in need of treatment for a selected condition. For example, the subject may be a subject suffering from a condition, may be symptomatic of a selected condition, or may be at risk of a selected condition. In some embodiments, in place of a subject, the neddylation inhibitors are administered to cultured cells.

Therapeutically Effective Amount. The methods of the invention encompass the administration of neddylation inhibitors in a therapeutically effective amount. In one measure, therapeutically effective amount is an amount of neddylation inhibitor that is sufficient to measurably reduce neddylation of proteins in a selected cell type. In another measure, a biologically or therapeutically effective amount is an amount of neddylation inhibitor that is sufficient to have a measurable therapeutic effect. In one measure, a therapeutic is the attainment of a specific physiological outcome or state, for example, any relevant outcome, such as reduced symptoms of a selected condition, improved organ or cellular function, and other physiological or health measures.

Treatment. The methods of the invention encompass the prevention or treatment of a selected condition. As used herein, “treatment” will encompass any number of therapeutic effects and outcomes with respect to a selected condition, including, for example: a reduction in the severity of symptoms of the condition; the inhibition of pathological processes underlying the condition; the reversal of pathological events or processes of the condition; halting or slowing the progression of the condition; or a reduction in morbidity and/or mortality associated with the condition. Treatment, as used herein, will further encompass prevention of an enumerated condition. As used herein, prevention will encompass any number of actions with respect to a selected condition, for example: preventing the onset of the condition; reducing the probability of the condition occurring; halting the further progression of the condition, ameliorating underlying physiological parameters that promote the condition, or any other preventative action. As used herein, treatment will further encompass enhancements of target cell or organ function, such as quantitatively or qualitatively improved function, for example, in certain implementations, improved function, restoring normal function, or maintaining function.

Immune Conditions. The scope of the invention encompasses the treatment of various immune conditions. The immune condition may be any condition wherein dysregulated immune processes are enabled, promoted, or mediated by neddylation processes. In one embodiment, the immune condition is a condition associated with the activity of NAE. In one embodiment, the immune condition is a condition associated with the activation, proliferation, or activity of immune cells. In one embodiment, the immune condition is a condition associated with the activation, proliferation, or activity of CD4+ T cells.

In a primary implementation, the methods of the invention are directed to the treatment of MS. MS treatment may encompass any treatment or prevention of MS, including decreasing the progression of MS, decreasing the severity and/or frequency of relapse, slowing or amelioration of MS symptoms, such as improving measures such Expanded Disability Status Scale score (EDSS), Multiple Sclerosis Severity Score, Multiple Sclerosis Functional Composite score (MSFC), global brain atrophy, grey matter atrophy, white matter atrophy, and retinal axonal degeneration

In another aspect, the scope of the invention encompasses a method of treating a demyelinating condition. In one embodiment, the demyelinating condition is MS. In other embodiments, the demyelinating disease may be Devic's disease, an inflammatory demyelinating disease, or an acute disseminated encephalomyelitis. The demyelinating condition may comprise a leukodystrophic disorder. The demyelinating condition may comprise a central nervous system neuropathy, central pontine myelinolysis, or progressive multifocal leukoencephalopathy. Treatment of such conditions encompasses any of curing a demyelinating condition; ameliorating symptoms associated with demyelination (e.g. nerve signal disruption, axonal damage, and neurodegeneration); slowing the progression of a demyelinating condition; preventing or delaying the onset of a demyelinating condition in an at-risk subject; preventing further loss of myelin; or restoring lost myelin.

In another aspect, the immune condition is any of a number of conditions mediated by immune cell activity. Exemplary immune conditions include, for example, any disease, condition, or dysfunction comprising inflammation or a self-immune process. Exemplary immune conditions include multiple sclerosis, arthritis (e.g., rheumatoid arthritis), inflammatory bowel disease, Crohn disease, lupus, autoimmune uveitis, type I diabetes, bronchial asthma such as lupus, retinitis, pancreatitis, cardiomyopathy, pericarditis, colitis, glomerulonephritis, lung inflammation, esophagitis, gastritis, duodenitis, ileitis, meningitis, encephalitis, encephalomyelitis, transverse myelitis, cystitis, urethritis, mucositis, lymphadenitis, dermatitis, hepatitis, and osteomyelitis.

Many neurodegenerative diseases are driven, exacerbated, or mediated by the activity of immune cells. In one aspect, the immune condition encompasses a neurodegenerative condition. Exemplary neurodegenerative disorders include multiple sclerosis, Parkinson's disease, Alzheimer's disease, Schizophrenia, myasthenia gravis, multiple sclerosis, microbial infections, head trauma and stroke, Pick's disease, dementia with Lewy bodies, Huntington disease, chromosome 13 dementias, Down's syndrome, cerebrovascular disease, Rasmussen's encephalitis, viral meningitis, NPSLE, amyotrophic lateral sclerosis, Creutzfeldt-Jacob disease, Gerstmann-Straussler-Scheinker disease, transmissible spongiform encephalopathies, ischemic reperfusion damage (e.g. stroke), brain trauma, microbial infection, chronic fatigue syndrome, Mild Cognitive Impairment; and movement disorders (including ataxia, cerebral palsy, choreoathetosis, dystonia, Tourette's syndrome, kernicterus), tremor disorders, leukodystrophies (including adrenoleukodystrophy, metachromatic leukodystrophy, Canavan disease, Alexander disease, Pelizaeus-Merzbacher disease), neuronal ceroid lipofucsinoses, ataxia telangectasia, or Rett Syndrome.

Neddylation Inhibitors. The methods of the invention encompass the administration of a neddylation inhibitor. A neddylation inhibitor may comprise any composition of matter which inhibits one or more steps of the neddylation pathway. The neddylation pathway may include any of:
processing of neural-precursor-cell-expressed developmentally down-regulated 8 (NEDD8) precursor to its activated form by deneddylase DEN1;
binding of APPBP1 (Amyloid Precursor Protein-Binding Protein 1) binds to the UBA3 (ubiquitin-like protein-activating enzyme 3) to form an activated E1 enzyme, such as NAE;
conjugation of NEDD8 to an E1 enzyme, such as NAE;
conjugation of NEDD8 to an E2 NEDD8 conjugating enzyme, such as UBE2F or UBE2M; and
conjugation of NEDD8 to a protein substrate (e.g. a cullin protein) by the action of an E3 ubiquitin ligase.

As described in the Examples, neddylation of various non-cullin target substrate proteins is implicated in the progression of immune disease. Accordingly, in some embodiments, the inhibition of neddylation encompasses inhibiting: the neddylation of VHL (von Hippel-Lindau tumor suppressor), the neddylation of AKIP1 (A-kinase interacting protein 1), and the neddylation of SMURF1 (SMAD specific E3 ubiquitin protein ligase 1).

Neddylation of the protein substrate causes any number of effects, including structural changes to the target protein that modulate its binding with any number of ligands, and recruitment of NEDD8-interacting proteins. In this way neddylation can act on numerous cellular processes, including immune cell processes. In one embodiment, the inhibition of neddylation is inhibition of neddylation in an immune cell. In one embodiment, the immune cell is a T-cell. In one embodiment, the immune cell is a CD4+ T Cell.

In one embodiment the neddylation inhibitor is an inhibitor of NAE. An NAF inhibitor is any composition of matter which inhibits the activity of NAE. For example, inhibition of NAE may encompass inhibition of one or more steps in NAE enzyme activity. NAE activity encompasses any of the following steps:
binding of a first ATP to NAE and a first NEDD8;
formation of a NEDD8-AMP intermediate:
binding of the NEDD8-AMP intermediate to the adenylation domain of NAE;
transfer of NEDD8 to the catalytic cysteine residue of NAE;
binding of a second ATP and a second NEDD8 to generate a second NEDD8-AMP intermediate.

In one embodiment, the neddylation inhibitor is pevonedistat, also known as MLN4924. Pevonedistat is an AMP mimetic that forms a stable adduct with NEDD8 in the NAF catalytic pocket, blocking further activity by the enzyme. Pevonedistat may be administered at any effective dosage, for example, at dosages of 1-50 mg/kg body area, for example 10-25 mg/kg, for example, by infusion for 1-50 administrations, for example, 1-5 administrations per week. In one embodiment, the neddylation inhibitor is a Pevonedistat derivative. Pevonedistat derivatives include variants of the MLN4924 structure which retain NAE inhibition activity. Exemplary derivatives are described, for example, in U.S. Pat. No. 8,980,850, entitled Administration of a NEDD8-activating enzyme inhibitor and hypomethylating agent, by Smith.

In one embodiment, the neddylation inhibitor is a composition described in U.S. Pat. No. 9,447,156, Methods and compositions for inhibiting neddylation of proteins by Monda et al.

In one embodiment, the neddylation inhibitor is a composition described in U.S. Pat. No. 9,850,214, Inhibitors of NEDD8-activating enzyme, by McCarron et al.

In one embodiment, the neddylation inhibitor is a composition described in United States Patent Application Publication Number 20190255052, entitled Inhibitors of E1 Activating Enzymes, by Langston et al.

The neddylation inhibitor may further comprise any inhibitor of processes in the neddylation pathway, including inhibitors of NEDD8 activation, UBE2F or UBE2M inhibitors, or inhibitors of E3 ligation enzymes active in neddylation, such as RBX1, RBX2, and DCUN1D1-5.

In one embodiment, the neddylation inhibitor is an agent, such as a nucleic acid construct, which inhibits the expression of one or more elements of the neddylation pathway, for example, the NAE1 gene, the UBA3 gene, the UBE2F or UBE2M genes, or an E3 ligation enzyme active in neddylation, such as RBX1, RBX2, and DCUN1D1-5. Exemplary nucleic acid constructs include siRNAs, siRNA, CRISPR-Cas9 or like constructs, TALENs, or other gene-expression targeting compositions, for example, which achieve target gene knock-down, target gene knockout, insertional mutagenesis, or post-translational degradation.

The neddylation inhibitors utilized in the methods of the invention may be formulated for efficient delivery by a selected route, for example, intravenous, intra-arterial, intraperitoneal, intrapulmonary, oral, inhalation, intravesicular, intramuscular, intra-tracheal, subcutaneous, intraocular, intrathecal, transmucosal, and transdermal delivery. Neddylation inhibitors may be formulated in combination with pharmaceutically acceptable excipients, carriers, diluents, release formulations and other drug delivery or drug targeting vehicles, as known in the art. In one embodiment, the neddylation inhibitors may be formulated as nanoparticles containing or functionalized with the selected active agent, for delivery by nanoparticle-based delivery methods. In one embodiment, the neddylation inhibitor composition comprises the selected therapeutic agent admixed with a polymeric material for timed release elution of the agent or to prevent premature digestion of the material in the digestive tract. In one embodiment, the neddylation inhibitor is coated onto an implant or drug eluting device

Exemplary Embodiments. In some embodiments, the scope of the invention encompasses: a neddylation inhibitor for use in a method of treating an immune condition, a neddylation inhibitor for use in a method of inhibiting neddylation in immune cells, a neddylation inhibitor for use in a method of inhibiting the activation, activity and/or proliferation of immune cells, in some embodiments, a neddylation inhibitor for use in a method of inhibiting the activation, activity, or proliferation of CD4+ T cells; in some embodiments, the immune condition is a condition mediated by activation, proliferation, or activity of immune cells, in some embodiments, the immune cells are CD4+ T cells; in some embodiments the immune condition is multiple sclerosis, in some embodiments the immune condition is a demyelinating condition, in some embodiments the immune condition is an autoimmune condition, in some embodiments, is a condition associated with dysregulated inflammatory responses, in some embodiments, the immune condition is a neurodegenerative condition; in some embodiments, the neddylation inhibitor is an inhibitor of any of an E1 NEDD8 activator, an E2 NEDD8 conjugator, or an E3 NEDD8 ligase, in some embodiments, the neddylation inhibitor is an NAE inhibitor, in some embodiments, the NAE inhibitor is pevonedistat, in some embodiments the NAE inhibitor is a pevonedistat derivative or variant; in some embodiments, the neddylation inhibitor is a nucleic acid construct or other agent which inhibits the expression (e.g. transcription or translation) of an E1 NEDD8 activating enzyme, an E2 NEDD8 conjugating enzyme, or an E3 NEDD8 ligase enzyme, in some embodiments, being an inhibitor of NAE1, UBE2F, UBE2M, RBX1, RBX2, or DCUN1D1-5.

In another aspect, the scope of the invention encompasses a method of treating an immune condition in a subject in need of treatment therefor by the administration to the subject of a therapeutically effective amount of a neddylation inhibitor; in some embodiments, the immune condition is a condition mediated by activation, proliferation, or activity of immune cells, in some embodiments, the immune cells are CD4+ T cells; in some embodiments, the immune condition is multiple sclerosis; in some embodiments, the treatment reduces the severity of multiple sclerosis, slows the progression of multiple sclerosis, or prevents the onset of multiple sclerosis; in some embodiments, the neddylation inhibitor inhibits the activity of NAE, in som embodiments, the neddylation inhibitor is pevonedistat.

### EXAMPLES

### Example 1 Targeted Transcriptomics Identifies Neddylation as a Novel Therapeutic Target in Multiple Sclerosis

Introduction. Multiple sclerosis is a chronic autoimmune condition of the central nervous system characterized by demyelination and neurodegeneration leading to permanent clinical disability. While it is clear that both genetic and environmental factors contribute to MS pathogenesis, its etiology remains elusive. With the exception of anti-B cell therapies, current immunomodulatory therapies are only partially effective. Over the past decade, genome-wide association studies (GWAS) identified 233 independent risk loci in MS, most of which harbor genes primarily expressed in immune cells such as T cells and monocytes. Previous gene expression studies used whole blood or peripheral blood mononuclear cells (PBMC) mostly searching for dysregulated MS pathogenic pathways and biomarkers of disease progression. However, this approach only detects the strongest signals due to highly variable gene expression across the complex mixture of cell types present in each sample. In order to increase the sensitivity of this analysis, performed cell-type-specific RNA-seq was performed for FACS-sorted specific immune cell populations, thus enhancing the signal-to-noise ratio.

EAE induction and neddylation inhibition in mice. Four weeks old female C57BL/6 mice were used. All mice were housed in closed caging systems and provided with standard irradiated chow diet, acidified water ad libitum and housed under a 12-hour light cycle. Pevonedistat (MLN4924) (Chemietek Inc., Indianapolis, Ind.) was dissolved in DMSO and further diluted in 30% of PEG300 and 5% of TWEEN in ddH2O at a concentration of 40 mg/mL and stored at −20° C. 7-8 week old mice were treated daily with 20 mg/kg Pevonedistat or vehicle (same concentration of buffer without Pevonedistat) starting at day −1. Mice were immunized subcutaneously on day 0 with 100 μg MOG35-55 emulsified in incomplete Freund's adjuvant supplemented with Mycobacterium tuberculosis followed by two intraperitoneal injections on Day 0 and Day 2 with 300 ng pertussis each. To minimize adverse events of Pevonedistat (excessive bleeding at injection site), mice were monitored for 15 minutes after immunization and additional care was provided as needed. Mice were scored daily on a 10-point scale in a blinded fashion as follows: 0, no deficit; 1, limp tail only; 2, limp tail and hind limb weakness; 3, complete hind limb paralysis; 4, complete hind limb paralysis and partial/complete forelimb paralysis; 5, death.

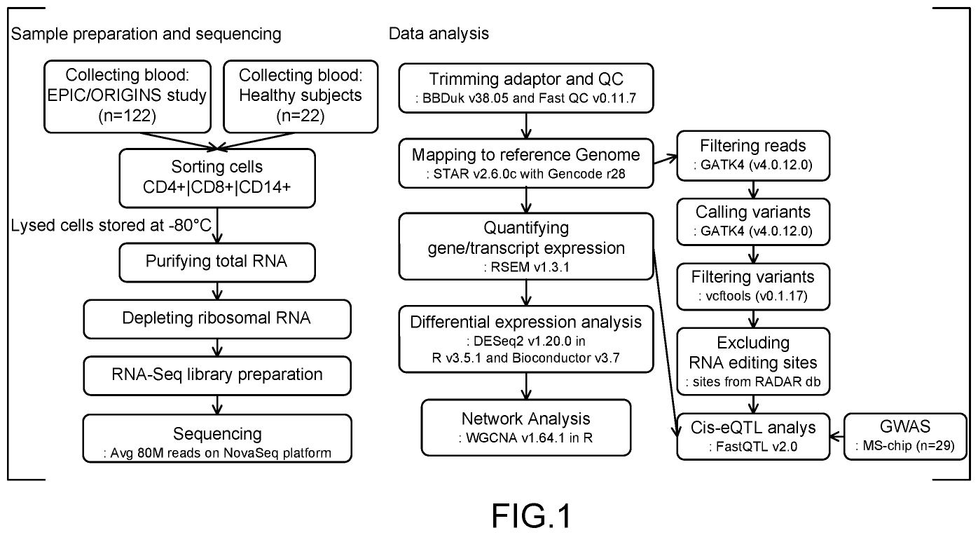

Results. Total RNA of FACS-sorted CD4+ T cells, CD8+ T cells, and CD14+ monocytes from 122 MS patients and 22 healthy subjects was sequenced. Most of the patients were newly diagnosed with MS or at an early stage of the disease highlighted by the short median disease duration (1 year) and median EDSS score (2.0). The complete experimental and analytical workflow is shown in FIG. 1. A PCA (principal component analysis) plot using all transcripts that passed QC in all samples showed that the gene expression was primarily clustered by cell types but not by disease status. T cells and monocytes were clearly separated by first principal component (PC1), and CD4+ and CD8+ T cells were mostly separated by second (PC2) and third components (PC3). A total of 122 treatment-naive patients were used for further analysis.

Differentially expressed genes (DEGs) (FDR 5%) were then searched for between treatment-naive patients and healthy subjects in each cell subset, and 464 DEGs in CD4+, 93 in CD8+, and 612 in CD14+ cells were identified. No gene expression differences across disease subtypes were observed.

On average, 36.6% of total DEGs were non-coding RNA and pseudogenes (ncRNA), and remarkably, 88.2% of the ncRNAs were down-regulated in MS patients. On the other hand, on average, 63% of protein-coding genes were up-regulated in CD4+ and CD14+ cells (37.9% in CD8+ cells). 73% of DEGs in CD4+ cells (n=341), 43% in CD8+ (n=40), and 83% in CD14+ (n=507) were significant only within each cell subset.

Differentially expressed genes in MS patients. 464 significant DEGs were found in CD4+ cells between MS and healthy subjects. The most significant down-regulated transcript was OMG (oligodendrocyte myelin glycoprotein) whose expression is typically restricted to the brain, where it plays a role in myelin formation. This gene was also significantly down-regulated in CD8+ and CD14+ cells. The transcript found here is one of 4 described for this gene, and contains a retained intron which results in a non-protein-coding sequence with a potential regulatory role as seen in other ncRNA families. A disease course comparison within treatment-naive patients identified only a small number of DEGs. Several other genes, including NAE1 (NEDD8 activating enzyme E1 subunit 1) were significantly up-regulated in CD4+ T cells from MS patients. NAE1 encodes a subunit of the NEDD8 activating enzyme (NAE), which forms a heterodimer with UBA3 and can activate the NEDD8 (NEDD8 ubiquitin-like modifier; neural precursor cell expressed developmentally down-regulated 8) conjugation pathway called neddylation.

Most DEGs in CD8+ cells were down-regulated in MS. Meanwhile, the largest number of DEGs was found in CD14+ cells (n=612). Specifically, SOCS3 was also significantly up-regulated. SOCS3 has been described as up-regulated in the Ml-like macrophage, an inflammatory monocyte/macrophage state. Up-regulation of other inflammatory state associated genes such as IL1b and CSF2RB was also found. Altogether, the data suggest that monocytes in MS patients are more polarized towards an inflammatory state.

27 significant DEGs were found whose expression overlapped between both CD4+ and CD8+ T cells, and only 16 transcripts were differentially expressed in all cell subsets. CDC42SE2 (CDC42 small effector 2) was most up-regulated in both T cell subsets but not in monocytes. CDC42 (cell division cycle 42), is a GTPase of the Rho subfamily, and regulates cell morphology, migration, endocytosis, and cell cycle progression. Most importantly, it has also been identified to play an important role in T cell development and migration into the central nervous system. These results suggest that migration and immunological synapse activity are increased in MS patients.

Search for eQTLs. In order to evaluate if gene expression was correlated with genetic susceptibility variants, significant DEGs were compared with the proximal gene list of IMSGC genome-wide effect region. One such gene was found in CD4+ T cells (CD37) and one in CD8+ T cells (VANGL2), while 12 genes (CD6, IL7R, NCF4, CD37, CCR4, CSF2RB, CD28, CD5, LCK, LEF1, TCF7, EPPK1) met this criteria in CD14+ cells. Although many genes from the IMSGC study are associated with T cell pathways, more IMSGC proximal genes were found from CD14+ monocytes. Those genes were associated with cell surface receptor signaling pathways including T cell receptor and immune response.

Next it was sought to identify eQTLs and proximal genes associated with the IMSGC effect region in a cell-type-specific manner. In total, the permutation-based test with GWAS genotypes revealed 23 significant eQTLs in CD4+ T cells, 22 in CD8+ T cells, and 64 (3 in MHC) in CD14+ monocytes. None of the significant DEGs were associated with eQTLs in the results from the GWAS genotype. With RNA-seq variants, a total of 414 significant eQTLs in CD4+ T cells was found (including 17 pairs in the MHC region), 329 in CD8+(21 in MHC), and 509 in CD14+(22 in MHC) in the treatment-naive MS patients. Fifty-three eQTLs overlapped across all cell subsets. Three DEGs (RP11-660L16.2, RP1-199J3.7, ULK4) were found among the eQTL genes in CD4+ T cells. However, we no genetic variants associated with genes from the neddylation pathway were found.

PPI and co-expression network analysis. In order to prioritize genes and identify related pathways, protein-protein interaction (PPI) network and weighted gene co-expression network analysis (WGCNA) were performed. A PPI network of DEGs using the STRING database in each cell subset was generated. In CD4+ cells, a main network containing 292 genes was identified. This network is significantly enriched in genes from “Acetylation” and “Ubl conjugation (conjugation of ubiquitin-like protein)” pathways. These terms are closely related to protein modification function (post-translational modification). Of note, NAE1 itself was included in the main network. Furthermore, enzyme subunits which associated with NAE1 such as ASB7 (ankyrin repeat and SOCS box containing 7), LRRC41 (leucine rich repeat containing 41), WDTC1 (WD and tetratricopeptide repeats 1), FBXL22 (F-box and leucine rich repeat protein 22) were found.

WGCNA revealed 6 co-expressed gene modules (4 up-regulated and 2 down-regulated) from CD4+ cells, 6 modules (3 up-regulated and 3 down-regulated) from CD8+ cells, and 5 modules (4 up-regulated and 1 down-regulated) from CD14+ cells. The “black” module was most correlated with disease status but not with other phenotypes among the up-regulated gene modules in the CD4+ T cell subset. In order to find major biological terms/functions of each module, functional annotation analysis was performed with gene lists from each module. A total of 299 genes were included in the black module, and many of them were related to immune cell function. For instance, RHOA was suggested as a central regulator in T cell response and a potential therapeutic target for MS in a recent study (Zhang et al., Lesional accumulation of RhoA(+) cells in brains of experimental autoimmune encephalomyelitis and multiple sclerosis. Neuropathol Appl Neurobiol 2008; 34: 231-40 and Manresa-Arraut A, et al., RhoA Drives T-Cell Activation and Encephalitogenic Potential in an Animal Model of Multiple Sclerosis. Front Immunol 2018; 9: 1235). The functional annotation analysis of this module yielded significantly enriched terms such as “Acetylation”, “Phosphoprotein”, and “Ubl conjugation”. Altogether, these results point towards post-translational modifications such as neddylation and ubiquitination pathways as important pathways in the regulation of CD4+ T cells in MS.

Given the prominent dysregulation of pathways involved in post-translational modifications and, in particular, the increased expression of NAE1 in MS, it was decided to further explore the neddylation pathway in more detail. Previous studies have shown that neddylation is required for T cell receptor (TCR)-mediated T cell functions (Mathewson et al. Neddylation plays an important role in the regulation of murine and human dendritic cell function. Blood 2013; 122: 2062-73; Jin et al., Neddylation pathway regulates T-cell function by targeting an adaptor protein Shc and a protein kinase Erk signaling. Proc Natl Acad Sci USA 2013; 110: 624-9; and Cheng et al. Neddylation contributes to CD4+ T cell-mediated protective immunity against blood-stage Plasmodium infection. PLoS Pathog 2018; 14: e1007440). Incidentally, dysregulation of other genes in this pathway was also found, including UBE2F (ubiquitin conjugating enzyme E2 F) and known substrate proteins of the neddylation pathway: VHL (von Hippel-Lindau tumor suppressor), AKIP1 (A-kinase interacting protein 1), and SMURF1 (SMAD specific E3 ubiquitin protein ligase 1). UBE2F encodes the NEDD8-conjugating enzyme E2 which catalyzes the transfer of NEDD8 from NAE to a substrate protein in the neddylation pathway. These results demonstrated that neddylation plays an important role in MS pathogenesis.

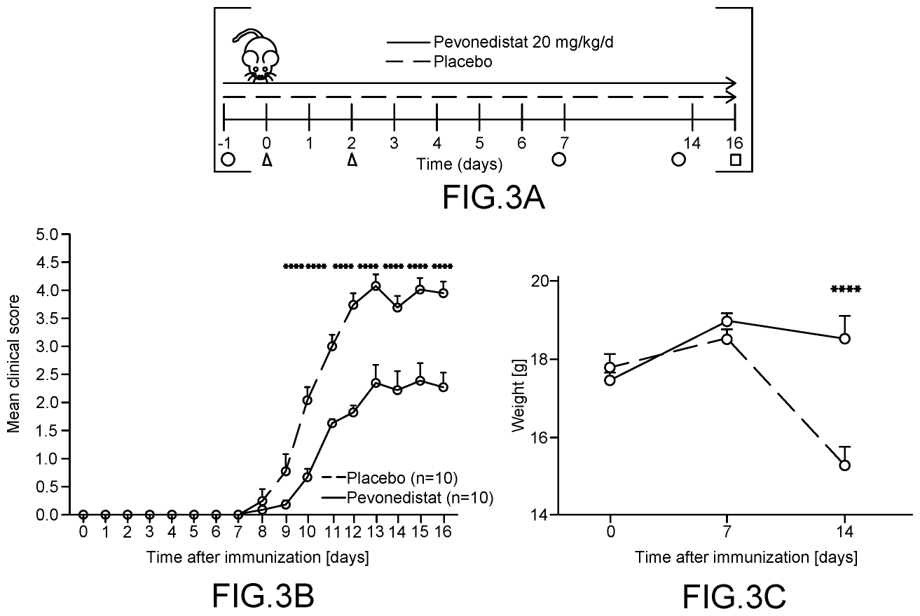

Inhibition of neddylation reduces EAE. Up-regulation of NAE1, a subunit of NAE (NEDD8 activating enzyme), which is essential for the ubiquitin-like post-translational modification pathway called neddylation and the significant enrichment of post-translational modification pathways in samples from MS subjects demonstrated the importance of neddylation in MS pathogenesis. Pevonedistat (MLN4924), which is a small molecule and first-in-class inhibitor of NAE was next used in experimental autoimmune encephalomyelitis (EAE), an established MS mouse model driven by a strong CD4+ T cell response (FIG. 3A). Although Pevonedistat was originally tested as a cancer therapy, recent studies proposed this agent as a potential therapeutic target for immune-related diseases due to the important role of neddylation in immune cell functions.

Treatment with Pevonedistat significantly reduced EAE severity compared to the placebo-treated group (FIG. 3B). This difference in disease severity was also reflected by steady weight in the Pevonedistat-treated animals during the disease course compared to a significant weight loss at peak of disease in the placebo group (FIG. 3C). Histological assessment of the CNS tissue at peak disease confirmed a significant reduction of demyelination in the Pevonedistat-treated group. Further, multifocal inflammatory infiltrates were observed in the EAE spinal cords of placebo-treated animals, while treatment with the drug resulted in a dramatic reduction in spinal cord inflammation as shown by decreased densities of Iba1+ myeloid cells and in particular CD3+ T cells in EAE mice.

Discussion Immune cells, especially T cells, are known to play an important role in MS pathogenesis. In the early stages of MS, these autoreactive T cells traffic to the CNS from peripheral blood where they trigger demyelination and cause neuro-axonal injury. Notably, T cells and macrophages are enriched in the brain and cerebrospinal fluid (CSF) of MS patients. In this work, we explored the cell-type-specific transcriptional landscape of CD4+ and CD8+ T cells and CD14+ monocytes in treatment-naive MS patients. The cell-type-specific transcriptome results indicated that CD14+ monocytes were the most dysregulated in MS among the three different immune cells.

Neddylation regulates protein function and activity including cullin-RING E3 ubiquitin ligase (CRL) activity. Similar to ubiquitination, neddylation is a cascade of 3 enzymatic processes by E1, E2, and E3 enzymes. NAE activates the process as an E1 enzyme by NEDD8 conjugation. CRLs in turn, associate with Skp2, forming the SCFskp2 complex which mediates degradation of numerous proteins by the ubiquitin proteasome system. One of the targets of this complex, which itself is regulated by neddylation, is Tob1, a tumor suppressor gene with activity in T cell proliferation, EAE, and potentially MS. Several E3 enzyme subunits that can be associated with more specific pathways following neddylation were also found. These E3 enzyme subunits might be important in a specific CD4+ T cell function of MS pathogenesis. Inhibition of neddylation in CD4+ T cells suppresses T cell proliferation and cytokine production by inhibiting the NF-κB pathway and increasing SOCS1 and SOCS3 expression, which in turn suppresses T cell function. The NF-κB pathway is important in immune cells' pro-inflammatory response. The neddylation pathway activates NF-κB via degradation of NF-κB inhibitor (IκB) by the ubiquitin proteasome system and/or neddylation of TRAF6 (TNF receptor associated factor 6) protein. Therefore, inhibition of neddylation also suppresses NF-κB activation. In the EAE model, CD4+ T cells activated against myelin protein infiltrate the CNS and cause neuroinflammation. The NAE inhibitor Pevonedistat (MLN4924) significantly reduced EAE severity with a dramatic reduction in spinal cord inflammation. These murine results indicate that the neddylation pathway plays a critical role in T cell activation and/or proliferation during EAE, and combined with the human RNA-seq data, this pathway is potentially implicated in human MS pathogenesis as well.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A neddylation inhibitor for use in a method of treating an immune condition.

2. The neddylation inhibitor of claim 1, wherein
the immune condition is a condition mediated by activation, proliferation, or activity of immune cells.

3. The neddylation inhibitor of claim 2, wherein
the immune cells are CD4+ T cells.

4. The neddylation inhibitor of claim 1, wherein
the immune condition is multiple sclerosis.

5. The neddylation inhibitor of claim 1, wherein
the treatment reduces the severity of multiple sclerosis, slows the progression of multiple sclerosis, or prevents the onset of multiple sclerosis.

6. The neddylation inhibitor of claim 1, wherein
the neddylation inhibitor inhibits the activity of NAE.

7. The neddylation inhibitor of claim 1, wherein
the neddylation inhibitor is pevonedistat.

8. A method of treating an immune condition in a subject in need of treatment therefor by the administration to the subject of a therapeutically effective amount of a neddylation inhibitor.

9. The method of claim 8, wherein
the immune condition is a condition mediated by activation, proliferation, or activity of immune cells.

10. The method of claim 9, wherein
the immune cells are CD4+ T cells.

11. The method of claim 8, wherein
the immune condition is multiple sclerosis.

12. The method of claim 8, wherein
the treatment reduces the severity of multiple sclerosis, slows the progression of multiple sclerosis, or prevents the onset of multiple sclerosis.

13. The method of claim 8, wherein
the neddylation inhibitor inhibits the activity of NAE.

14. The method of claim 8, wherein
the neddylation inhibitor is pevonedistat.
